(19) United States
(12) Patent Application Publication
(10) Pub. No.: US 2024/0197159 A1
(43) Pub. Date: Jun. 20, 2024

(54) Scene Adaptive Endoscopic Illuminator

(71) Applicant: KARL STORZ Imaging, Inc., Goleta, CA (US)

(72) Inventor: George E. Duckett, III, Castaic, CA (US)

(73) Assignee: KARL STORZ Imaging, Inc., Goleta, CA (US)

(21) Appl. No.: 18/085,437

(22) Filed: Dec. 20, 2022

(51) Int. Cl. A61B 1/06; A61B 1/00

(52) U.S. Cl. CPC A61B 1/0684; A61B 1/00096; A61B 1/0655

(57) ABSTRACT

Endoscopic illuminator devices and methods are provided with an adaptive illumination profile. An endoscopic illuminator includes a first illumination element adapted for coupling first illumination light into a light input of an endoscope for illuminating a central area of a field of view of the endoscope. A second illumination element is adapted for coupling second illumination light to the light input for illuminating a peripheral area of the field of view, arranged relative to the first illumination element such that the first illumination light enters the light input at a substantially different angle from the first illumination light, and having an illumination level adjustable separately from that of the first illumination element.

### TECHNICAL FIELD OF THE INVENTION

The invention relates to light sources or illuminators for endoscopes and other scopes.

### BACKGROUND OF THE INVENTION

FIG. 1 illustrates an endoscopic imaging system with an endoscopic illuminator according to the prior art. An endoscope 10 includes an endoscopic shaft member 20 attached to a handle or camera head 30. Illumination source 40 may be integrated as depicted within a scope controller 80, which also powers and communicates with endoscope 10 through cable 70. Scope controller 80 provides an image signal to a display 90. Light collected by an objective lens at the distal end of the endoscopic shaft 20 is alternatively captured by a, usually distally placed, image sensor within the shaft, or relayed down the length of the shaft via a relay lens system to an image sensor located within the camera head. The distal end of the shaft 20 is inserted into an otherwise inaccessible space, such as body cavity accessed through a small incision. An illumination source 40 produces illumination light that is directed into a light guide 50 which carries the illumination light to a light post 60, where the light guide is coupled with another light guide (such as a fiber bundle) that carries the illumination to the distal end of the shaft 20, where it is then able to illuminate a scene within the inaccessible space. As an alternate to utilizing an imaging endoscope 10 to provide illumination, an endoscopic light source might simply embody a light pipe comprising a shaft which may be inserted through a separate incision from that of an imaging endoscope.

Current endoscopic illuminators provide illumination with a constant angular profile. Such constant profiles are suboptimal for several reasons. When an endoscope looks down a lumen (a tubular cavity) it is frequently the case that the sides of the lumen, which are close to the endoscope, are over-illuminated while the center of the scene is under-illuminated. Conversely, while looking at a flat or convex scene, it is often the case that the edges of the image are under-illuminated while the center is over-illuminated. A typical endoscopic illuminator does not allow for adjustment in the light source to optimize the spatial illumination.

One solution is to use image processing, either high-dynamic range (HDR) imaging techniques or other localized brightness adjustments techniques, to increase the apparent brightness of dark regions of the detected image. However, such techniques have disadvantages in that that they either reduce resolution, require higher frame rates, or amplify noise in addition to the amplified signal, and require more image processing power.

What is needed are devices and methods to enable improved illumination of endoscopic imaging.

### SUMMARY OF THE INVENTION

It is an object of the invention to provide an endoscopic illumination with improved illumination profiles. It is a further object of the invention to provide improved control of an illumination profile. Another object of the invention is to provide such improvements in a manner compatible with various light guides for various endoscopic systems.

According to a first aspect of the invention, an endoscopic illuminator includes first and second illumination elements. The first illumination element adapted is for coupling first illumination light into a light input of an endoscope for illuminating a central area of a field of view of the endoscope. The second illumination element adapted is for coupling second illumination light to the light input for illuminating a peripheral area of the field of view. The second illumination element is arranged relative to the first illumination element such that the second illumination light enters the light input at a substantially different angle from the first illumination light, and having an illumination level adjustable separately from that of the first illumination element.

According to some implementations of the first aspect, the first and second illumination elements each include a light source and a light directing element arranged for directing light from the light source to the light input at their respective different angles. The light directing elements may be lenses. At least one of the light directing elements may include a parabolic or elliptical mirror. The light directing elements of the first and second illumination elements may be arranged for collecting light from their respective light source and directing a converging field of light into the light input centered around the first or second angle, respectively.

According to some implementations of the first aspect, the first and second illumination elements are adapted to couple light into a light input selected from the group comprising: an input of a light post of the endoscope, a light port of the endoscope, an input of a light cable of the endoscope, an input of a light pipe of the endoscope, and an optical element for coupling to a light input for an endoscope.

According to some implementations of the first aspect, a controller is also included, the controller coupled to the first and second illumination elements and operable to automatically adjust the illumination levels of the first and second illumination elements to improve the evenness of illumination over a field of view of the endoscope. The illumination at the edges of the endoscopic field of view may be automatically decreased relative to the illumination at the center of the field of view when the endoscope images along a lumen, and the illumination at the edges of the endoscopic field of view may be automatically increased relative to the illumination at the center of the field of view when the endoscope images a flat or convex scene. The optical axis of the illumination element providing illumination to the center of the endoscopic field of view is oriented at a slight angle with respect to the longitudinal axis of the light input into which the illumination is coupled.

According to some implementations of the first aspect, at least one additional illumination element is included.

According to some implementations of the first aspect, at least two of the illumination elements provide illumination to the periphery of a field of view of the endoscope.

According to some implementations of the first aspect, the illumination elements are LEDs.

According to a second aspect of the invention, a method is given for providing illumination for an endoscope. The method includes coupling first illumination light into a proximal end of a light guiding element for an endoscope centered along a first angle relative to the light guiding element. The method includes illuminating a central area of a field of view of the endoscope from a distal end of the light guiding element with the first illumination light. While coupling the first illumination light, second illumination light is coupled into the light guiding element centered along a second angle substantially different from the first angle. The method includes illuminating a peripheral area of the field of view with the second illumination light, and adjusting an illumination level of the second illumination light relative to that of the first illumination light to improve evenness of illumination in the field of view. According to some implementations of the second aspect, the method includes detecting a first illumination level in the central area of the field of view and detecting a second illumination level in the peripheral area of the field of view. Based on the first and second illumination levels, the illumination level of the first or second illumination light is automatically adjusted.

According to some implementations of the second aspect, coupling the first illumination light into the proximal end of the light guiding element includes directing the first illumination light at the light guide element with a first lens directed along the first angle. Coupling the second illumination light into the proximal end of the light guiding element may include directing the second illumination light at the light guide element with a second lens directed along the second angle.

According to a third aspect of the invention, an endoscopic imaging system includes an endoscope, a light source, and a controller. The endoscope includes an endoscope comprising a shaft, a light guide extending along at least a portion of the shaft to a distal end of the shaft, and a light input. The light source a first illumination element providing first illumination light into the light input, which is emitted by the light guide to illuminate a central area of a field of view of the endoscope. The light source includes a second illumination element providing second illumination to the light input and arranged relative to the first illumination element such that the second illumination enters the light input at a substantially different angle from the first illumination, which is emitted by the light guide to illuminate a peripheral area of the field of view. The second illumination element has an illumination level adjustable separately from that of the first illumination element. The controller is coupled to the first and second illumination elements and operable to adjust at least one of the first and second illumination elements to improve the evenness of illumination over an endoscopic field of view.

According to some implementations of the third aspect, the first and second illumination elements each comprise a light source and a light directing element arranged for directing the illumination from the light source to the light input at their respective different angles.

According to some implementations of the third aspect, the light directing elements are lenses.

According to some implementations of the third aspect, at least one of the light directing elements comprises a parabolic or elliptical mirror.

According to some implementations of the third aspect, the controller is operable to automatically adjust at least one of the illumination elements based on a first illumination level detected in the central area of the field of view and a second illumination level detected in the peripheral area of the field of view.

These and other features of the invention will be apparent from the following description of the preferred embodiments, considered along with the accompanying drawings.

### BRIEF DESCRIPTION OF THE DRAWINGS

The present invention will become more fully understood from the detailed description given herein and the accompanying drawings which are given by way of illustration only, and thus are not limitative of the present invention, and wherein:

FIG. 1 illustrates a conventional endoscopic imaging system including an endoscope connected by a light guide to an illumination source;

FIG. 2 illustrates in diagram form a portion of an endoscopic Illuminator 100 according to some embodiments;

FIG. 3 illustrates in optical diagram form endoscopic illuminator according to some embodiments;

FIG. 4 illustrates in optical diagram form endoscopic illuminator according to some additional embodiments;

FIG. 5 illustrates in optical diagram form endoscopic illuminator according to some additional embodiments;

FIG. 6 illustrates in optical diagram form endoscopic illuminator including an additional illumination element according to some additional embodiments;

FIG. 7 illustrates in optical diagram form endoscopic illuminator including a tapered light guide according to some embodiments;

FIG. 8 shows a flowchart of a process for operating an endoscopic illuminator according to some embodiments;

FIG. 9 shows a chart of an angular illumination profile for an endoscopic illuminator according to some embodiments;

FIG. 10 shows a chart depicting a number of combined angular illumination profiles for an endoscopic illuminator according to some embodiments; and

FIG. 11 shows a block diagram of an imaging system including an image capture device and an endoscope device having an improved endoscopic illuminator as described above.

### DETAILED DESCRIPTION OF EXAMPLE EMBODIMENTS

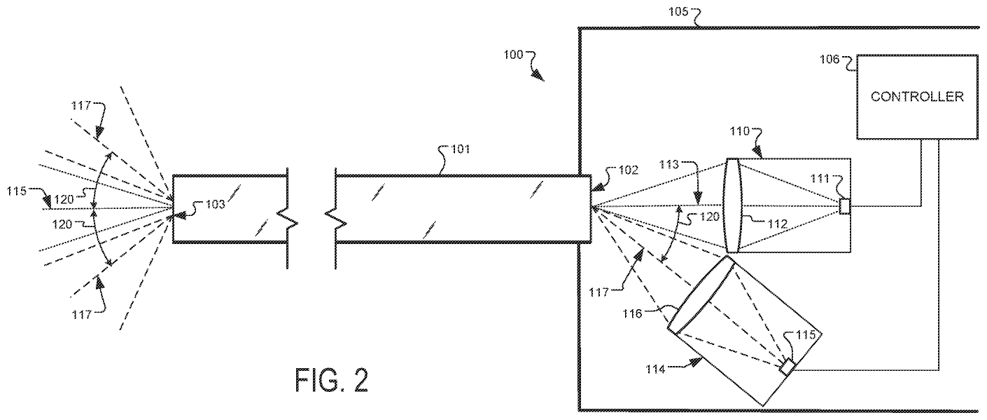

FIG. 2 illustrates in diagram form a portion of an endoscopic illuminator 100 according to some embodiments. The depicted portion of endoscopic illuminator 100 generally includes a light guide 101 and an endoscopic illuminator 105 coupled to light guide 101 for providing illumination to a proximal end of light guide 101 at a light input 102. The illumination light is shown leaving light guide 101 at a distal end 103 for illuminating a field of view of an endoscope.

Light guide 101 may be any suitable light guide for an endoscopic system, including a light cable or light pipe, for receiving illumination light and carrying it to the endoscope, where it is typically coupled to a light post. In such embodiments, the light post is optically coupled into a second light guide to carry light to the scope's distal end 103 to illuminate the scope's field of view. Generally, such a light guide includes one or more light carrying members such as fiber optic cables, fiber optic bundles, plastic optical fibers, or light pipe elements. In the usual case where the light transmitting components of the light guide maintain a constant diameter over the length of the light guide, the light entering the light guide at light input 102 leaves the light guide at substantially the same angle, with some small amount of dispersion caused by diffraction and the bending of the light fibers. Light input 102 is generally a proximal planar face of light guide 101, such as a proximal face of a fiber optic bundle, fiber optic cable, or other type of light guide. Light guide 101 typically includes an attachment mechanism (not shown) such as a plug or receptacle housing for attaching light guide 101 to endoscopic illuminator 105 and positioning light input 102 in a desired position relative to the illumination sources. Light input 102 may embody any suitable light input of a light cable or input of a light pipe of an endoscope. The light cable can then be connected to the light post (as described above) of an endoscope. Alternatively, the light cable (generally removably attached) may be an integral part of the endoscope, obviating the need for a coupling light post. In another embodiment, the endoscopic illuminator may be integrated with the endoscope. Light guide 101 may include more than one light carrying element coupled to each other. For example, a light conditioning element may be positioned in endoscopic illuminator 100 presenting a proximal face to provide light input 102, and a distal face coupled to a light cable for an endoscope or an intervening element. In some embodiments where the endoscopic illuminator 100 is sufficiently small and mobile, such as with a battery powered, portable unit, it may be connected directly to the light post 60 of an endoscope, and the light post coupler may serve as the light input 102. User interface controls, such as buttons on, or in electronic communication with, the attached endoscopic illuminator 100 may be used to select a desired illumination profile, as described below.

Endoscopic illuminator 105 includes a housing generally indicated by label 105, which may be in the form of a light box, a portable light source module, or a portion of a larger control unit for an endoscope in which endoscopic illuminator 105 is integrated. The depicted endoscopic illuminator 105 includes a controller 106, a first illumination element 110, and a second illumination element 114. More illumination elements may be used in other embodiments, as further described below. First illumination element 110 includes a light source 111, a light directing element 112 arranged for collecting light from light source 111 and directing a converging cone of light centered around an angle indicated by line 113 to light input 102, and may include a housing generally indicated by arrow 110. Second illumination element 114 includes a light source 115, a light directing element 116 optically arranged for collecting light from light source 115 and directing a converging cone of light centered around an angle indicated by line 117 to light input 102, and may include a housing generally indicated by arrow 114. Light sources 111 and 115 are both connected to controller 106 to separately and independently control the illumination level of each light source (111, 115), as further discussed below. Light sources 111 and 115 may be any suitable light sources which allow for control of the illumination level, such as LEDs, for example.

As depicted, in this embodiment, angle 113 is aligned with a central optical axis of light guide 101 for illuminating a central area of the endoscope's field of view. In other embodiments, angle 113 may be slightly offset from the central optical axis. Second illumination element 114 is adapted for coupling second illumination light to light input 102 for illuminating a peripheral area of the field of view, and is optically arranged relative to the first illumination element such that the second illumination light enters the light input at an angle (shown by line 117) substantially different angle from the first illumination light, as shown by the angular arc 120, and has an illumination level adjustable independently from that of the first illumination element 110. In this embodiment, light directing elements 116 are lenses, but in other embodiments they may include other suitable light directing elements or a combination of elements such as, for example, parabolic or elliptical mirrors, or portions of a larger singular parabolic or elliptical mirror, as further described below.

As shown in FIG. 2, in this embodiment the illumination light from each of the individual illumination elements 110 and 114 is generally not a unidirectional ray but a field of illumination that is focused by light directing elements 112 and 116. The first illumination light leaves its light source 111 as a diverging light field as illustrated by the dotted lines, impinges on light directing elements 112 (here, a lens), and is focused toward light input 102 along a central axis indicated by line 113. Preferably, the focal point of light directing element 112 is at or near the surface of light input 102 as depicted by the converging dotted lines on the drawing, providing the benefit that most of the light from the illumination element and directed by the directing element will enter the light input 102. A similar arrangement for second illumination element 114 is depicted by the larger dashed lines, with the second illumination light shown as a light field focused by light directing element 116 toward light input 102 along a central axis indicated by lines 117.

The resulting illumination light emitted at distal end 103 of light guide 101 is depicted on the left of the diagram. The first illumination light is depicted leaving distal end 103 of light guide 101 to illuminate the central area of the field of view. The central area is generally circular as indicated by the dotted lines at diverging angles around line 113. The second illumination light leaves distal end 103 to illuminate the peripheral area of the endoscope's field of view. Due to the optical properties of light guide 101, while light enters light input 102 at an angle from one side, the exiting light from distal end 103 is generally a ring encircling the central portion of the field of view to illuminate the peripheral portion, with the original field of light being emitted as a ring as indicated by the dashed lines, with a central radius indicated by the lines 117 at angle 120 surrounding the central optical axis of the distal end 103 at the depicted line 113. While the two fields of illumination light are shown as separate, preferably the edges overlap to provide continuous a continuous illumination field from the combined illumination of the first and second illumination lights, which has an adjustable distribution as further discussed below.

In operation, controller 106, coupled to the first illumination element 110 and second illumination element 114, is operable to automatically adjust the illumination levels of the first and second illumination elements 110 and 114 to improve the evenness of illumination over a field of view of the endoscope. For example, the illumination at the edges of the endoscopic field of view may be automatically decreased relative to the illumination at the center of the field of view when the endoscope images along a lumen, and automatically increased relative to the illumination at the center of the field of view when the endoscope images a flat or convex scene.

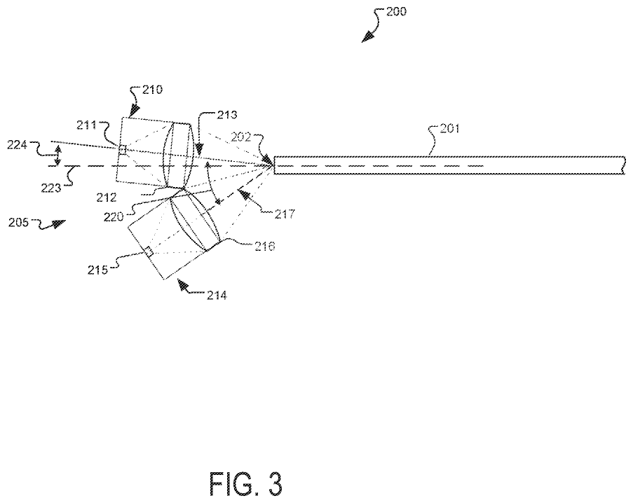

FIG. 3 illustrates in optical diagram form endoscopic illuminator 200 according to some embodiments. The depicted portion of endoscopic illuminator 200 generally includes a light guide 201 and an endoscopic illuminator 205 coupled to light guide 201 for providing illumination to a proximal end of light guide 201 at a light input 202. Endoscopic illuminator 205 is suitable for use with various types of endoscopes and light guides as discussed with respect to FIG. 2, and provides illumination light from a distal end of light guide 201 as described with respect to FIG. 2. Endoscopic illuminator 205 includes a first illumination element 210 and a second illumination element 214, generally constructed like those of FIG. 2.

First illumination element 210 includes a light source 211, and a light directing element 212 optically arranged for collecting light from light source 211 and directing a converging cone of light centered around an angle indicated by line 213 to light input 202. Second illumination element 214 includes a light source 215 and a light directing element 216 optically arranged for collecting light from light source 215 and directing a converging cone of light centered around an angle indicated by line 217 to light input 202.

In this embodiment, the illumination light of first illumination element 210 has a central optical axis along which the illumination light travels, indicated by the angle of line 213, which is arranged at a slight angular offset from a central optical axis 223 of light guide 201, indicated by arc 224. For example, an angular offset of less than 10 degrees may be considered slight, or in some embodiments an angular offset between 3 degrees and 8 degrees, or 3 degrees and 10 degrees may be used. This angular offset generally provides a more dispersed illumination in the central area of the endoscope's field of view than embodiments in which the first illumination element is aligned with optical axis 223. In particular this offset prevents a gap in the illumination profile between the angles illuminated by the first illumination element 210 and the second illumination element 214.

Second illumination element 214 is adapted for coupling second illumination light to light input 202 for illuminating a peripheral area of the field of view, and is arranged relative to the first illumination element such that the second illumination light enters the light input at an angle (shown by line 217) substantially different angle from the first illumination light, as shown labelled by angular arc 220, and has an illumination level adjustable separately from that of the first illumination element 212.

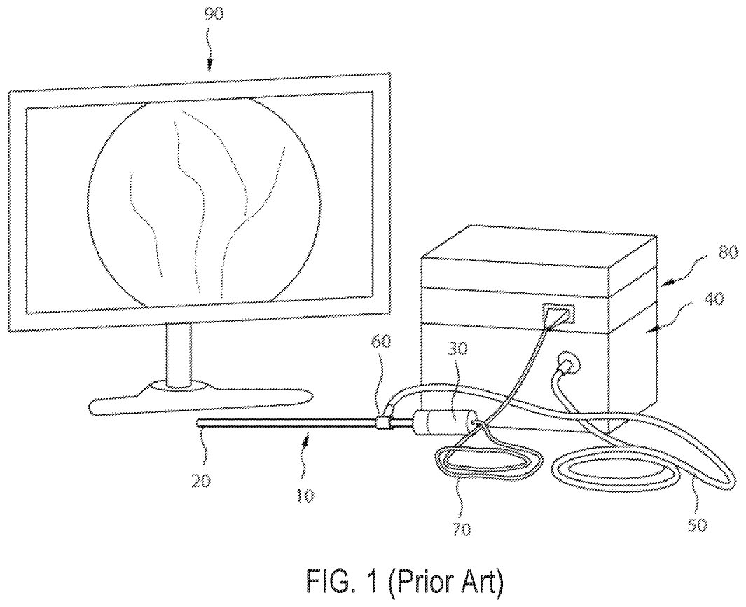

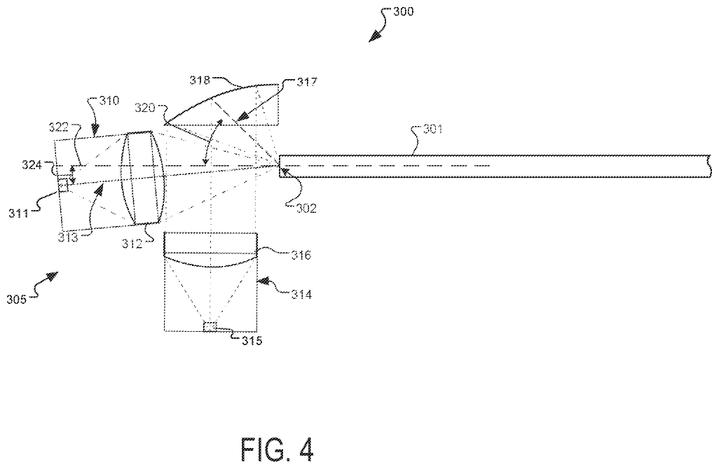

FIG. 4 illustrates in optical diagram form endoscopic illuminator 300 according to some embodiments. The depicted portion of endoscopic illuminator 300 generally includes a light guide 301 and an endoscopic illuminator 305 coupled to light guide 301 for providing illumination to a proximal end of light guide 301 at a light input 302. Endoscopic illuminator is suitable for use with various types of endoscopes and light guides as discussed with respect to FIG. 2 and provides illumination light from a distal end of light guide 301 as described with respect to FIG. 2. The depicted endoscopic illuminator 305 includes a first illumination element 310 generally constructed like that of FIG. 1, and a second illumination element 314, including a light guide element constructed with a parabolic or elliptical mirror 318, as further discussed below.

First illumination element 310 includes a light source 311 and a light directing element 312 arranged for directing light from light source 311 to light input 302 at angle generally indicated by line 313.

Second illumination element 314 includes a light source 315 and light directing elements 316 and 318 arranged for directing light from light source 315 to light input 302 at angle generally indicated by line 317. Light directing element 316 is a lens which directs diverging illumination light leaving light source 315 to a field of generally parallel light rays directed at light directing element 318. For example, light directing element 316 may be any suitable optical element for achieving this effect, for example a convex-plano lens or a convex-convex lens. Light directing element 318 is a curved reflecting element such as a parabolic mirror or an elliptical mirror and is optically arranged for redirecting and focusing the second illumination light toward light input 302 at the central angle indicated by line 317.

In this embodiment, the illumination light of first illumination element 310 has a central optical axis along which the illumination light travels, indicated by the angle of line 313, which is arranged at a slight angular offset from a central optical axis 322 of light guide 301, indicated by arc 324, which is sized similarly to the angular offset of arc 224 of FIG. 3.

Second illumination element 314 is adapted for coupling second illumination light to light input 302 for illuminating a peripheral area of the field of view, and is arranged relative to the first illumination element such that the second illumination light enters the light input at an angle (shown by line 317) that is substantially different from the angle (shown by line 313) of the first illumination light. The labelled angular arc 320 represents the deviation from the central optical axis 322 of the central rays of the second illumination element. The second illumination element 314 has an illumination level adjustable separately from that of the first illumination element 310.

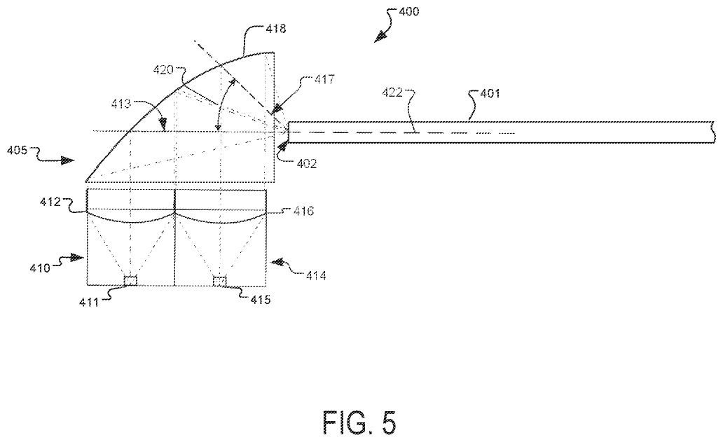

FIG. 5 illustrates in optical diagram form endoscopic illuminator 400 according to some embodiments. The depicted portion of endoscopic illuminator 400 generally includes a light guide 401 and an endoscopic illuminator 405 coupled to light guide 401 for providing illumination to a proximal end of light guide 401 at a light input 402. Endoscopic illuminator 405 is suitable for use with various types of endoscopes and light guides as discussed with respect to FIG. 2 and provides illumination light from a distal end of light guide 401 as described with respect to FIG. 2. Endoscopic illuminator 405 includes a first illumination element 410, and a second illumination element 414, and a common light guide element 418 constructed with a parabolic or elliptical mirror, as further discussed below.

First illumination element 410 includes a light source 411 and a light directing element 412 optically arranged for directing light from light source 411 to a portion of light directing element 418 and then to light input 402 at angle generally indicated by line 413. Second illumination element 414 includes a light source 415, a light directing elements 416, and a portion of light directing element 418 arranged for directing light from light source 415 to light input 402 at angle generally indicated by line 417.

Light directing element 416 is a lens which directs light from the diverging illumination light leaving light source 415 to a field of generally parallel light rays directed at light directing element 418. Light directing element 412 is constructed similarly. For example, light directing elements 412 and 416 may be any suitable optical element for achieving this effect, for example a convex-plano lens or a convex-convex lens. Light directing element 418 is a curved reflecting element such as a parabolic mirror or an elliptical mirror and is optically arranged for redirecting and focusing the first illumination light toward light input 402 and redirecting and focusing second illumination light toward light input 402 at the central angle indicated by line 417. In this embodiment, different portions of light directing element 418 are used to redirect and focus the first and second illumination light. In other embodiments, light directing element 418 may be replaced with two separate light directing elements such as parabolic or elliptical mirrors.

In this embodiment, the illumination light of first illumination element 410 has a central optical axis along which the illumination light travels, indicated by the angle of line 413, which is arranged parallel to a central optical axis 422 of light guide 401.

Second illumination element 414 is adapted for coupling second illumination light to light input 402 for illuminating a peripheral area of the field of view, and is arranged relative to the first illumination element such that the second illumination light enters the light input at an angle (shown by line 417) substantially different angle from the first illumination as shown by the labelled angular arc 420, and has an illumination level adjustable separately from that of the first illumination element 410. In this embodiment, the angular offset at arc 420 is achieved by the combination of the relative positions of light directing elements 412, 416, and 418. This embodiment has the benefit of permitting first and second illumination elements 410, 414 to be mounted on the same printed circuit board and may also simplify the alignment of the illumination with the light input.

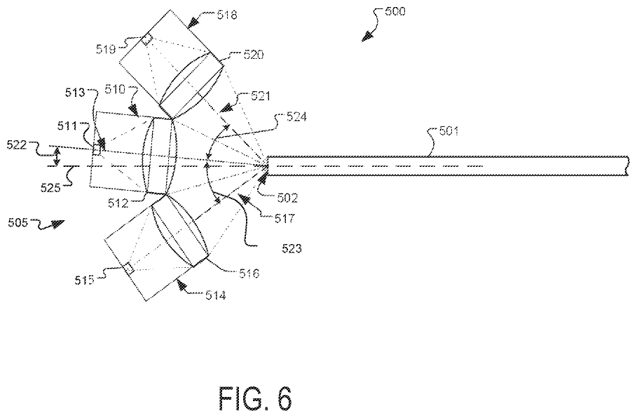

FIG. 6 illustrates in optical diagram form endoscopic illuminator 500 according to some embodiments. The depicted portion of endoscopic illuminator 500 generally includes a light guide 501 and an endoscopic illuminator 505 coupled to light guide 501 for providing illumination to a proximal end of light guide 501 at a light input 502. Endoscopic illuminator 505 is suitable for use with various types of endoscopes and light guides as discussed with respect to FIG. 2 and provides illumination light from a distal end of light guide 501 as described with respect to FIG. 2. Endoscopic illuminator 505 includes a first illumination element 510, a second illumination element 514, and a third illumination element 518, generally constructed like those of FIG. 2.

First illumination element 510 includes a light source 511, and a light directing element 512 optically arranged for directing light from light source 511 to light input 502 at angle generally indicated by line 513. Second illumination element 514 includes a light source 515, and a light directing element 516 optically arranged for directing light from light source 515 to light input 502 at angle generally indicated by line 517. Third illumination element 518 includes a light source 519, and a light directing element 520 optically arranged for directing light from light source 519 to light input 502 at angle generally indicated by line 521.

In this embodiment, the illumination light of first illumination element 510 has a central optical axis along which the illumination light travels, indicated by the angle of line 513, which is arranged at a slight angular offset from a central optical axis 525 of the distal portion of light guide 501, indicated by arc 522. For example, an angular offset of less than 10 degrees may be considered slight, or in some embodiments an angular offset between 3 degrees and 8 degrees, or 3 degrees and 10 degrees may be used. This angular offset generally provides a more dispersed illumination in the central area of the endoscope's field of view than embodiments in which the first illumination element is aligned with optical axis 525.

Second illumination element 514 is adapted for coupling second illumination light to light input 502 for illuminating a peripheral area of the field of view, and is arranged relative to the first illumination element such that the second illumination light enters the light input at an angle (shown by line 517) substantially different angle from the first illumination light, as shown by the labelled angular arc 523, and has an illumination level adjustable separately from that of first illumination element 510.

Third illumination element 518 is adapted for coupling third illumination light to light input 502 for illuminating a peripheral area of the field of view, and is arranged relative to the first illumination element such that the second illumination light enters the light input at an angle (shown by line 521) substantially different angle from the first illumination light, as shown by the labelled angular arc 524, and has an illumination level adjustable separately from that of the first illumination element 510 and second illumination element 514.

As can be understood from the diagram, the central illumination angle provided by second illumination element 514 shown by line 517 and arc 523 is also different from the central illumination angle provided from third illumination element 518 shown by line 521 and arc 524. Third illumination element 518 is at a steeper angle relative to the central axis because it has the angle of arc 522 added, while the angle of line 517 has the angle of arc 522 subtracted. Thus, the second and third illumination elements 514 and 518 illuminate different but overlapping portions of the periphery of the endoscope's field of view as their illumination light exits the distal end of light guide 501, providing a broader “ring” of illumination. Further, the use of two illumination elements for the peripheral illumination provides greater adjustability of the illumination and enables the potential to provide increased illumination of peripheral regions than would be possible with a single illumination element. In other embodiments, a similar arrangement may be employed, but without the offset depicted by arc 522. Instead, first illumination element 510 may be aligned with optical axis 525. The arrangement of second and third illumination elements may be symmetrical about optical axis 525 or may provide different angles (at arcs 523 and 524).

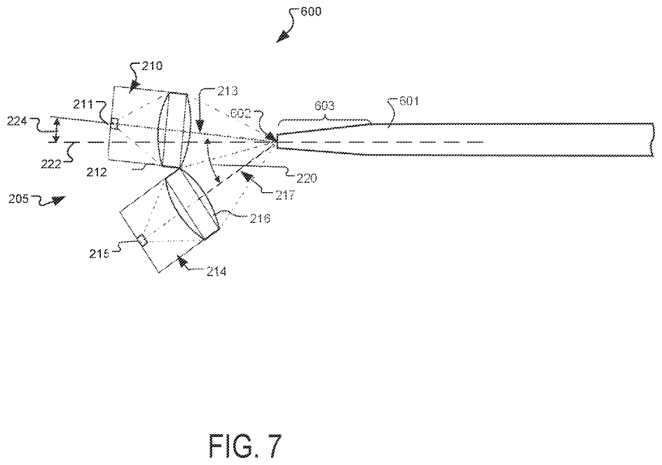

FIG. 7 illustrates in optical diagram form endoscopic illuminator 600 including a tapered light guide 601 according to some embodiments. The depicted portion of endoscopic illuminator 600 generally includes a light guide 601 and an endoscopic illuminator 205 coupled to light guide 601 for providing illumination to a proximal end of light guide 601 at a light input 602. Endoscopic illuminator 205 is described above with respect to FIG. 2 and will not be repeated here.

In this embodiment, tapered light guide 601 is a tapered light pipe or fiber optic taper with a cylindrical portion generally indicated by 601, and a tapered end portion 603 presenting light input 602 at the distal end of tapered light guide 601. As can be seen, the surface area of light input 602 is smaller than that of light input 202 of FIG. 2 for a similarly sized light guide. Such a tapered end portion offers the designer a further degree of freedom in the conditioning of the light. The tapered end portion may be integrated with the light guide or a separate element to which the light guide may be attached to couple the illumination light. For example, when the input 602 is a smaller diameter than the output at the distal end of the light guide, the angles with which light departs the light guide will be reduced on the output compared to the input. Such a design degree of freedom may be very valuable in cases where the mechanical components are large, and it is difficult to bring first illumination element 210 and second illumination element 214 close enough together to produce a desired end illumination profile. Additionally, if the output taper is smaller than the input 602, the angular spread will be increased.

While the use of endoscopic illuminator 205 of FIG. 2 is shown with tapered light guide 601, other embodiments of an endoscopic illuminator as described herein may also be used with a tapered light guide.

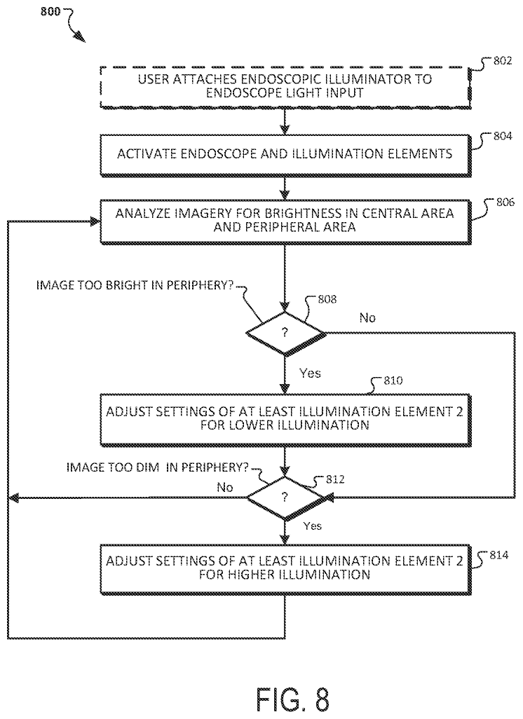

FIG. 8 shows a flowchart 800 of a process for operating an endoscopic illuminator according to some embodiments. The process is suitable for use with all embodiments of an endoscopic illuminator herein and is generally conducted by an electronic controller and control unit coupled to the endoscope. The process may be used in combination with other known illumination control features as, for example, automatic adjustment of overall image brightness. While the process steps are shown in a linear order here, generally they may be conducted in parallel, and may be conducted periodically, continuously, or in an object oriented control algorithm in which changes in brightness trigger certain control actions. The particular order of steps is therefore not limiting and is shown for illustrative purposes only.

The process starts at block 802 where a user attaches the endoscopic illuminator to the light input of an endoscope. For embodiments in which the endoscopic illuminator is integrated into the endoscope, this step is not used. At block 804, a user activates the endoscope and activates the endoscopic illuminator.

As shown at block 806, while the scope is in use, an image processor analyzes imagery from the scope to determine brightness in a central area of the scope field of view, and separately in a peripheral area. At block 808, the process checks if the image is too bright in the periphery. The determination may be made by measuring an overall brightness characteristic in the peripheral area and comparing it against a threshold brightness. Such a condition may exist, for example, if the scope is imaging along a lumen such as a colon (during a colonoscopy) or other tubular cavity, and the second illumination element as described in embodiments above is illuminating at or near the brightness of the first illumination element. In such a case, the walls of the lumen appear in the periphery and are closer to the endoscope tip than the any elements in the central area of the field of view, and so appear brighter. If so, the process goes to block 810 where it automatically adjusts the illumination setting of the second illumination element to reduce illumination, the first illumination element to increase the illumination to the central area, or adjust both illumination elements accordingly. Block 810 may also include adjusting the illumination of first illumination element to be higher, depending on its level and the overall image brightness, for example. If more than one illumination element is used for the periphery, more than one illumination element may be adjusted lower at block 810. If the condition is not present at block 808, the process goes to block 812.

At block 812, the process checks whether the imagery is too dim in the periphery. Such a condition may exist, for example, if the scope tip leaves a lumen and enters a larger cavity or changes orientation or position. If not, the process returns to block 806 where it continues to analyze imagery. If so, the process goes to block 814 where it adjusts the settings of the second illumination element for higher illumination, and then proceeds to block 806. Block 814 may also include adjusting the illumination of first illumination element to be lower, depending on its level and the overall image brightness, for example.

The particular implementation of increasing or decreasing illumination at each illumination element may change depending on the type of illumination element used, and may include, for example, increasing or decreasing a pulse-width of a pulse with modulated signal supplying an LED of the illumination element, or increasing or decreasing a voltage or current supplied to the illumination element.

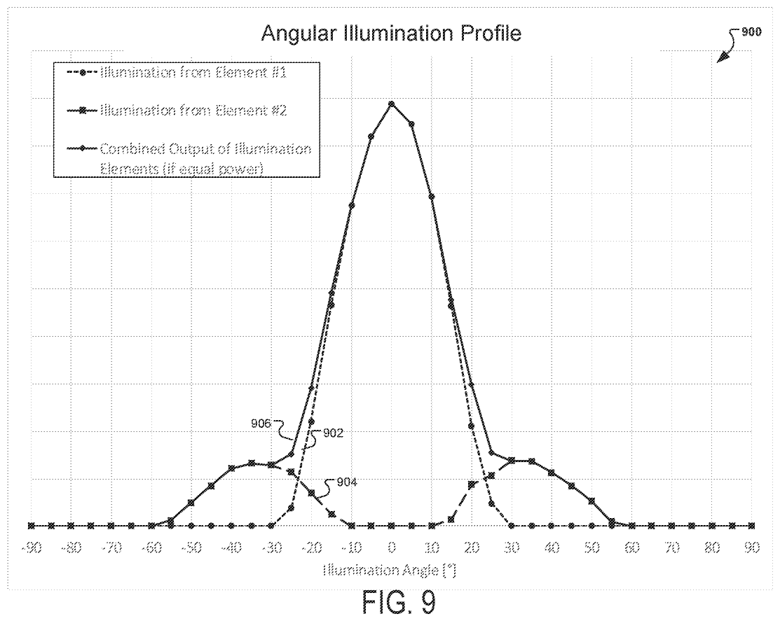

FIG. 9 shows a chart 900 of an angular illumination profile for an endoscopic illuminator according to some embodiments, such as the embodiment shown in FIG. 2. The vertical shows the illumination level, and the horizontal axis shows the angle of view as measured from directly perpendicular to the imaging lens or imager at the scope tip.

As can be seen, a first illumination profile 902 shows an angular illumination profile of a first illumination element, which emits light that is then directed, as described above, toward the center of the endoscope field of view. The illumination level or brightness drops off quickly from a peak in the center to a relatively low level at 25 degrees away from the center.

A second illumination profile 904 shows the angular illumination profile of a second illumination element as described above. As can be seen, there are two peaks on either side which illustrate the “ring” of light provided toward the image periphery by the second illumination element. This profile is low in the center and higher on each side with a peak around 35 degrees in this example.

A third profile 906 shows the combined illumination profile of both illumination elements. The particular illumination levels are shown merely as an example to illustrate the profile and change as the illumination levels are adjusted to change the shape of the overall profile. This profile might be one selected for illumination down a lumen where a lower intensity of light is desired at the peripheral regions and a higher intensity in the center of the image.

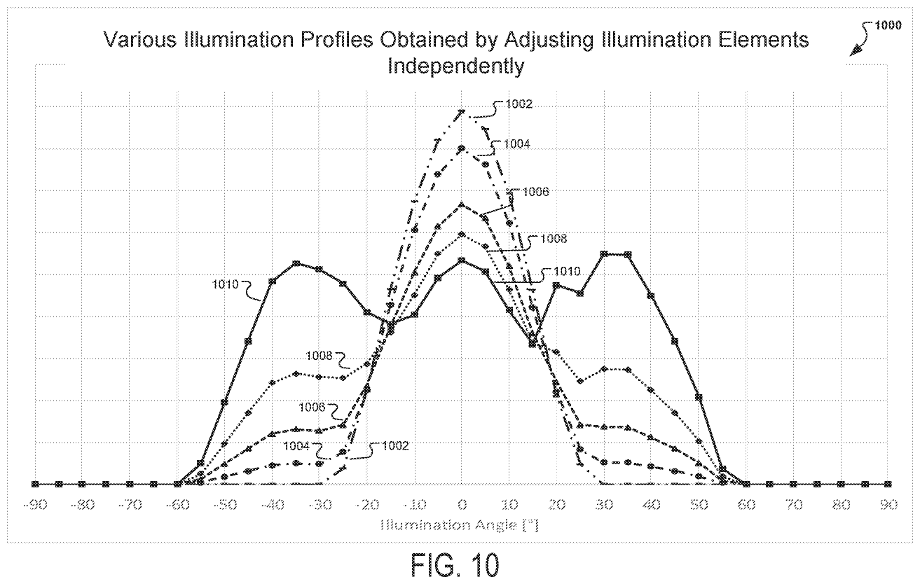

FIG. 10 shows a chart 1000 depicting a number of combined angular illumination profiles for an endoscopic illuminator according to some embodiments. Again, the vertical shows the illumination level, and the horizontal axis shows the angle of view as measured from directly perpendicular to the imaging lens or imager at the scope tip. The different profiles depict different combinations of illumination settings for the first and second illumination elements.

A first illumination profile 1002 results from a configuration in which the first illumination element is at its highest level illumination output, while the second illumination element is at its lowest level illumination output, i.e., turned off.

A second illumination profile 1004 results from a configuration in which the first illumination element has a high level illumination output, while the second illumination element has a low level illumination output.

A third illumination profile 1006 results from a configuration in which the first illumination element has a medium level illumination output, while the second illumination element has a medium level illumination output.

A fourth illumination profile 1008 results from a configuration in which the first illumination element has a high level illumination output, while the second illumination element has a medium-low level illumination output.

A fifth illumination profile 1010 results from a configuration in which the first illumination element has a low level illumination output, while the second illumination element has a high level illumination output. This sort of uniform illumination profile is useful, for example, when an observed scene or an object of interest is flat or convex.

As can be understood, the particular number of profiles and number of illumination levels presented are merely examples, and a typical embodiment includes more levels of illumination available for each illumination element. In operation, an endoscopic illuminator such as endoscopic illuminator 105 of FIG. 2 may be used with an adaptive illumination control method like that of FIG. 8 to adjust between various illumination profiles, or, alternatively, the profiles may be adjusted manually for a specific illumination profile configuration desired and selected by a user. For example, in the process of FIG. 8, at block 808 the illumination profile in one example scenario might be illumination profile 1010 when the process enters block 808. This profile has a relatively high illumination level in the peripheral area of the field of view (corresponding to the side lobes of the illumination profile), and when the scope is oriented along a lumen, such a profile would typically have too much brightness in the periphery at block 808. As a result of the adjustment at block 810, the process may adjust the profile to be 1008 or 1006, through one or more adjustment steps.

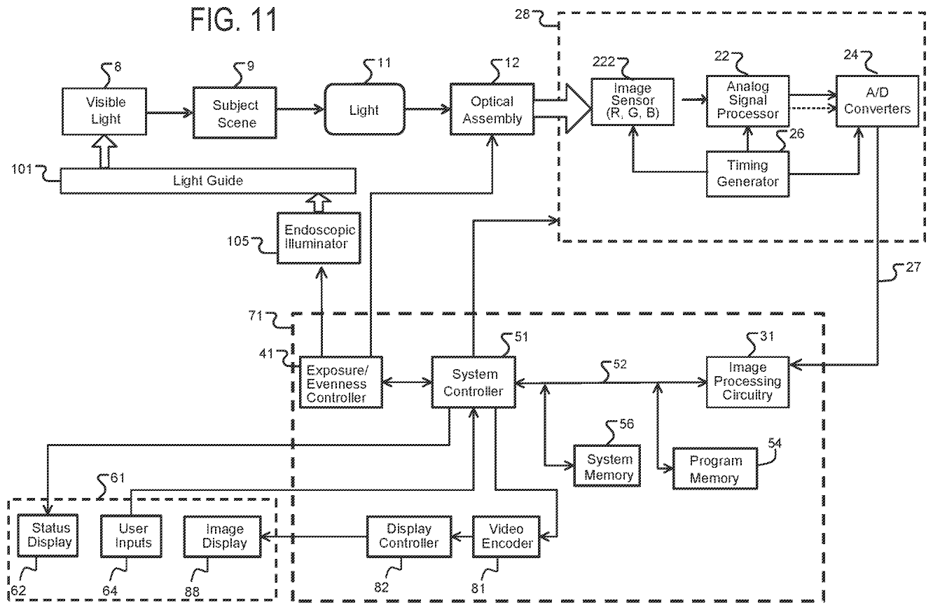

FIG. 11 shows a block diagram of an imaging system including an image capture device and an endoscope device having an improved endoscopic illuminator as described above. The endoscopic illuminators disclosed herein are applicable to more than one type of device enabled for image capture, for example: endoscopes, borescopes, and other medical scopes.

As shown in the diagram of an endoscopic imaging system, endoscopic illuminator 105 provides light to a light input of light guide 101, which is usually coupled to a light post of an endoscope, which in turn channels the light through the light guide of the endoscopic shaft to its distal end to illuminate the subject scene 9 with illumination light. Endoscopic illuminator 105 may be implemented with any of the various embodiments shown herein, such as endoscopic illuminators 205, 305, 405, 505, and 605, as well as other suitable embodiments. As shown in the drawing, light 11 reflected, scattered, or emitted from the subject scene is captured by an optical assembly 12, where the light is focused to form an image at a solid-state image sensor or sensors 222.

Optical assembly 12 generally includes one or more lenses and may include optical relay elements. Portions of the optical assembly may be embodied in a camera head or other first optical device, while other portions are in an endoscope or other scope device, or the optical assembly 12 may be contained in a single imaging device. Image sensor 222 (which may include separate R, G, and B sensor arrays) converts the detected light to an electrical signal by integrating charge for each picture element (pixel).

The image sensor 222 may be an active pixel complementary metal oxide semiconductor sensor (CMOS APS) or a charge-coupled device (CCD).

The total amount of light 11 reaching the image sensor 222 is regulated by the endoscopic illuminator 105 intensity, the optical assembly 12 aperture, and the time for which the image sensor 222 integrates charge. An exposure/evenness controller 41 performs an illumination profile control process such as that described with respect to FIG. 8, as well as controls exposure by responding to the amount of light available in the scene given the intensity and spatial distribution of digitized signals corresponding to the intensity and spatial distribution of the light focused on image sensor 222.

Exposure/evenness controller 41 may also control the optical assembly 12 aperture, and indirectly, the time for which the image sensor 222 integrate charges.

Analog signals from the image sensor 222 are processed by analog signal processor 22 and applied to analog-to-digital (A/D) converter 24 for digitizing the analog sensor signals. The digitized signals each representing streams of images or image representations based on the data, are fed to image processor 31 as image signal 27.

Image processing circuitry 31 includes circuitry performing digital image processing functions to process and filter the received images as is known in the art. Image processing circuitry performs analysis of the central area and peripheral area of the endoscope field of view to determine brightness levels used in controlling the illumination profile, for example, according to the process of FIG. 8. Such circuitry is known in the art and will not be further described here.

Timing generator 26 produces various clocking signals to select rows and pixels and synchronizes the operation of image sensor 222, analog signal processor 22, and A/D converter 24. Image sensor assembly 28 includes the image sensor 222, analog signal processor 22, A/D converter 24, and timing generator 26. The functional elements of the image sensor assembly 28 can be fabricated as a single integrated circuit as is commonly done with CMOS image sensors or they can be separately-fabricated integrated circuits.

The system controller 51 controls the overall operation of the image capture device based on a software program stored in program memory 54. This memory can also be used to store user setting selections and other data to be preserved when the camera is turned off.

System controller 51 controls the sequence of data capture by directing exposure/evenness controller 41 to set the light 8 intensity and distribution of the endoscopic illuminator 105, the optical assembly 12 aperture, and controlling various filters in optical assembly 12 and timing that may be necessary to obtain an image stream. A data bus 52 includes a pathway for address, data, and control signals.

Processed image data are continuously sent to video encoder 81 to produce a video signal. This signal is processed by display controller 82 and presented on image display 88. This display is typically a liquid crystal display backlit with light-emitting diodes (LED LCD), although other types of displays are used as well. The processed image data can also be stored in system memory 56 or other internal or external memory device.

The user interface 61, including all or any combination of image display 88, user inputs 64, and status display 62, is controlled by a combination of software programs executed on system controller 51. User inputs typically include some combination of typing keyboards, computer pointing devices, buttons, rocker switches, joysticks, rotary dials, or touch screens. The system controller 51 manages the graphical user interface (GUI) presented on one or more of the displays (e.g., on image display 88). In particular, the system controller 51 will typically have a mode toggle user input (typically through a button on the endoscope or camera head itself, but possibly through a GUI interface), and in response transmit commands to adjust image processing circuitry 31 based on predetermined setting stored in system memory.

Image processing circuitry 31 is one of three programmable logic devices, processors, or controllers in this embodiment, in addition to a 51 and the exposure/evenness controller 41. Image processing circuitry 31, controller 51, exposure/evenness controller 41, system and program memories 56 and 54, video encoder 81 and display controller 82 may be housed within camera control unit (CCU) 71.

CCU 71 may be responsible for powering and controlling endoscopic illuminator 105, image sensor assembly 28, and/or optical assembly 12. In some versions, a separate front end camera module may perform some of the image processing functions of image processing circuitry 31.

Although this distribution of imaging device functional control among multiple programmable logic devices, processors, and controllers is typical, these programmable logic devices, processors, or controllers can be combinable in various ways without affecting the functional operation of the imaging device and the application of the invention. These programmable logic devices, processors, or controllers can comprise one or more programmable logic devices, digital signal processor devices, microcontrollers, or other digital logic circuits. Although a combination of such programmable logic devices, processors, or controllers has been described, it should be apparent that one programmable logic device, digital signal processor, microcontroller, or other digital logic circuit can be designated to perform all of the needed functions. All of these variations can perform the same function and fall within the scope of this invention.

As used herein the terms “comprising,” “including,” “carrying,” “having” “containing,” “involving,” and the like are to be understood to be open-ended, that is, to mean including but not limited to. Any use of ordinal terms such as “first,” “second,” “third,” etc., in the claims to modify a claim element does not by itself connote any priority, precedence, or order of one claim element over another, or the temporal order in which acts of a method are performed. Rather, unless specifically stated otherwise, such ordinal terms are used merely as labels to distinguish one claim element having a certain name from another element having a same name (but for use of the ordinal term).

The foregoing has outlined rather broadly the features and technical advantages of the invention in order that the detailed description of the invention that follows may be better understood. It should be appreciated by those skilled in the art that the conception and specific embodiments disclosed may be readily utilized as a basis for modifying or designing other structures for carrying out the same purposes of the invention. It should also be realized by those skilled in the art that such equivalent constructions do not depart from the scope of the invention as set forth in the appended claims.

Although the invention and its advantages have been described in detail, it should be understood that various changes, substitutions and alterations can be made herein without departing from the scope of the invention as defined by the appended claims. The combinations of features described herein should not be interpreted to be limiting, and the features herein may be used in any working combination or sub-combination according to the invention. This description should therefore be interpreted as providing written support, under U.S. patent law and any relevant foreign patent laws, for any working combination or some sub-combination of the features herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the invention, processes, machines, manufacture, compositions of matter, means, methods, or steps, presently existing or later to be developed that perform substantially the same function or achieve substantially the same result as the corresponding embodiments described herein may be utilized according to the invention. Accordingly, the appended claims are intended to include within their scope such processes, machines, manufacture, compositions of matter, means, methods, or steps.

1. An endoscopic illuminator comprising:
a first illumination element adapted for coupling first illumination light into a light input of an endoscope for illuminating a central area of a field of view of the endoscope; and
a second illumination element adapted for coupling second illumination light to the light input for illuminating a peripheral area of the field of view, arranged relative to the first illumination element such that the second illumination light enters the light input at a substantially different angle from the first illumination light, and having an illumination level adjustable separately from that of the first illumination element.

2. The endoscopic illuminator of claim 1 wherein the first and second illumination elements each comprise a light source and a light directing element arranged for directing light from the light source to the light input at their respective different angles.

3. The endoscopic illuminator of claim 2 wherein the light directing elements are lenses.

4. The endoscopic illuminator of claim 2, wherein at least one of the light directing elements comprises a parabolic or elliptical mirror.

5. The endoscopic illuminator of claim 2, wherein the light directing elements of the first and second illumination elements are arranged for collecting light from their respective light source and directing a converging field of light into the light input centered around the first or second angle, respectively.

6. The endoscopic illuminator of claim 1 wherein the first and second illumination elements are adapted to couple light into a light input selected from the group comprising: an input of a light post of the endoscope, a light port of the endoscope, an input of a light cable of the endoscope, an input of a light pipe of the endoscope, and an optical element for coupling to a light input for an endoscope.

7. The endoscopic illuminator of claim 1 further comprising a controller coupled to the first and second illumination elements and operable to automatically adjust the illumination levels of the first and second illumination elements to improve the evenness of illumination over a field of view of the endoscope.

8. The endoscopic illuminator of claim 7 wherein the illumination at the edges of the endoscopic field of view is automatically decreased relative to the illumination at the center of the field of view when the endoscope images along a lumen, and wherein the illumination at the edges of the endoscopic field of view is automatically increased relative to the illumination at the center of the field of view when the endoscope images a flat or convex scene.

9. The endoscopic illuminator of claim 7 wherein the optical axis of the illumination element providing illumination to the center of the endoscopic field of view is oriented at a slight angle with respect to the longitudinal axis of the light input into which the illumination is coupled.

10. The endoscopic illuminator of claim 1 further comprising at least one additional illumination element.

11. The endoscopic illuminator of claim 10 wherein at least two of the illumination elements provide illumination to the periphery of a field of view of the endoscope.

12. The endoscopic illuminator of claim 1 wherein the illumination elements are LEDs.

13. A method of providing illumination for an endoscope, comprising:
coupling first illumination light into a proximal end of a light guiding element for an endoscope centered along a first angle relative to the light guiding element;
illuminating a central area of a field of view of the endoscope from a distal end of the light guiding element with the first illumination light;
while coupling the first illumination light, coupling second illumination light into the light guiding element centered along a second angle substantially different from the first angle;
illuminating a peripheral area of the field of view with the second illumination light; and
adjusting an illumination level of the second illumination light relative to that of the first illumination light to improve evenness of illumination in the field of view.

14. The method of claim 13, further comprising:
detecting a first illumination level in the central area of the field of view and detecting a second illumination level in the peripheral area of the field of view; and
based on the first and second illumination levels, automatically adjusting the illumination level of the first or second illumination light.

15. The method of claim 13, further wherein:
coupling the first illumination light into the proximal end of the light guiding element comprises directing the first illumination light at the light guide element with a first lens directed along the first angle; and
coupling the second illumination light into the proximal end of the light guiding element comprises directing the second illumination light at the light guide element with a second lens directed along the second angle.

16. An endoscopic imaging system comprising:
an endoscope comprising a shaft, a light guide extending along at least a portion of the shaft to a distal end of the shaft, and a light input;
a light source comprising:
a first illumination element providing first illumination light into the light input, which is emitted by the light guide to illuminate a central area of a field of view of the endoscope; and
a second illumination element providing second illumination to the light input and arranged relative to the first illumination element such that the second illumination enters the light input at a substantially different angle from the first illumination, which is emitted by the light guide to illuminate a peripheral area of the field of view, the second illumination element having an illumination level adjustable separately from that of the first illumination element;
a controller coupled to the first and second illumination elements and operable to adjust at least one of the first and second illumination elements to improve the evenness of illumination over an endoscopic field of view.

17. The endoscopic imaging system of claim 16 wherein the first and second illumination elements each comprise a light source and a light directing element arranged for directing the illumination from the light source to the light input at their respective different angles.

18. The endoscopic imaging system of claim 17 wherein the light directing elements are lenses.

19. The endoscopic imaging system of claim 17, wherein at least one of the light directing elements comprises a parabolic or elliptical mirror.

20. The endoscopic imaging system of claim 17, wherein the controller is operable to automatically adjust at least one of the illumination elements based on a first illumination level detected in the central area of the field of view and a second illumination level detected in the peripheral area of the field of view.
